(19) United States
(12) Patent Application Publication
(10) Pub. No.: US 2025/0064781 A1
(43) Pub. Date: Feb. 27, 2025

(54) METHODS OF TREATING, AMELIORATING, AND/OR PREVENTING STRESS-RELATED DISORDER

(71) Applicant: Yale University, New Haven, CT (US)

(72) Inventors: Amy Arnsten, New Haven, CT (US); Min Wang, New Haven, CT (US); Dibyadeep Datta, New Haven, CT (US)

(21) Appl. No.: 18/724,520

(22) PCT Filed: Dec. 22, 2022
(86) PCT No.: PCT/US22/82197

§ 371 Date: Jun. 26, 2024

Related U.S. Application Data
(60) Provisional application No. 63/293,874, filed on Dec. 27, 2021.

(51) Int. Cl. A61K 31/404; A61K 31/145

(52) U.S. Cl. CPC A61K 31/404; A61K 31/145

(57) ABSTRACT

In one aspect, described herein is a method of treating, ameliorating, and/or preventing a stress-related disorder in a subject in need thereof. In certain embodiments, the method includes administering to the subject a therapeutically effective amount of a α1A-adrenergic receptor subtype selective inhibitor.

### CROSS-REFERENCE TO RELATED APPLICATION

The present application claims priority under 35 U.S.C. § 119(e) to U.S. Provisional Patent Application No. 63/293,874, filed Dec. 27, 2021, which is incorporated herein by reference in its entirety.

### BACKGROUND

Reduction in the prefrontal cortical top-down control in the brain is known to occur in post-traumatic stress disorder (PTSD), anxiety disorders, as well as agitation/disruptive behaviors associated with dementia.

Stress increases norepinephrine (NE) release in the brain, and NE actions at alpha-1-adrenoceptors (also referred to as “α1-adrenergic receptors” or “alpha-1 adrenergic receptors” herein) contribute to the reduced top-down control and working memory mediated by the prefrontal cortex and the strengthened emotional responses (e.g., anxiety) by the amygdala. Elevated NE alpha-1-adenoceptor stimulation has been linked to the stress responses, including stress-induced weakening of prefrontal cortical top-control and working memory.

Due to the link between the elevated NE alpha-1-adenoceptor stimulation and the stress response, such as the stress-induced weakening of prefrontal cortical top-control and working memory, alpha-1-adrenoceptor antagonists have been used, or experimented on, for treating stress-related disorders.

For example, the alpha-1-adrenoceptor antagonist, prazosin, has been used to treat post-traumatic stress disorder (PTSD) and is being on clinical trial for treating disruptive behaviors in Alzheimer's Disease, which are believed to involve reduced prefrontal cortical top-down control. However, prazosin does not always work, as evidenced by the varied response of PTSD patients to prazosin, as well as some failed clinical trials (Peskind et al., N Engl J Med. 2018 Feb. 8;378(6):507-517, McCall et al., J Clin Psychopharmacol. 2018 Dec;38(6):618-621). Furthermore, the hypotensive and sedative side effects of prazosin can preclude the use of the compound in, for example, elderly patients with benign prostatic hyperplasia (BPH).

Another alpha-1-adrenoceptor antagonist, doxazosin, has been considered as a better alternative in treating stress-related disorders recently, as doxazosin has a longer half-life in vivo than prazosin. However, doxazosin shares similar shortcomings with prazosin, to various degrees.

There is thus a need in the art for novel methods of treating the stress-related disorders. Such methods should be more effective and have less adverse side effects than the currently used treatment methods. The present invention fulfills this need.

### SUMMARY

In some aspects, the present invention is directed to the following non-limiting embodiments.

In some embodiments, the present invention is directed to a method of treating, ameliorating, and/or preventing a stress-related disorder in a subject in need thereof.

In some embodiments, the method comprising administering to the subject a therapeutically effective amount of a selective α1-adrenergic receptor antagonist compound.

In some embodiments, the compound has a larger inhibition constant (pKi) for the α1A-adrenergic receptor subtype (α1A-AR) than for the α1B-adrenergic receptor subtype (α1B-AR) and/or α1D-adrenergic receptor subtype (α1D-AR).

In some embodiments, the compound has an inhibition constant (pKi) for the α1A-adrenergic receptor subtype (α1A-AR) that is 0.3 units or higher than for the α1B-adrenergic receptor subtype (α1B-AR) and/or α1D-adrenergic receptor subtype (α1D-AR).

In some embodiments, the selective α1A-adrenergic receptor antagonist compound comprises at least one selected from the group consisting of RS-17053, silodosin, 5-methyl-urapidil and tamsulosin.

In some embodiments, the stress-related disorder comprises a stress-induced prefrontal cortical dysfunction.

In some embodiments, the stress-related disorder is at least one selected form the group consisting of post-traumatic stress disorder (PTSD), an anxiety disorder, and an agitation/disruptive/aggressive behavior associated with dementia.

In some embodiments, the compound is administered by at least one route selected from the group consisting of oral, nasal, rectal, intravaginal, parenteral, buccal, sublingual, and topical.

In some embodiments, a dosage of the selective α1-adrenergic receptor antagonist administered to the subject is about 0.001 mg/kg or higher.

In some embodiments, a dosage of the selective α1-adrenergic receptor antagonist administered to the subject is about 1 mg/kg or lower.

In some embodiments, the subject is a mammal.

In some embodiments, the subject is a human.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIG. 1 depicts the performance in the spatial working memory task of monkeys exposed to stress or vehicle and administered with 0.1 mg/kg-1 mg/kg of RS17053 (RS17) or vehicle (VEH). Reversal of stress-induced cognitive deficits with the selective alpha1A-AR antagonist, RS17053. Stress is known to impair the higher cognitive functions that rely on the prefrontal cortex in humans, monkeys and rodents. In this experiment, rhesus monkeys exposed to a pharmacological agent that induces a stress response were impaired in performing a spatial working memory task that relies on the prefrontal cortex (VEH+STRESS). However, if the monkeys received pretreatment with RS17053 prior to the stressor, their cognitive performance was protected from stress exposure, performing as well (or even better) than under control, nonstress conditions. Results represent mean+SEM percent correct on the spatial working memory task. * significantly different from VEH+VEH; ** significantly different from VEH+STRESS; VEH=vehicle. In certain embodiments, the selective alpha1-AR antagonist RS17053 blocks the impairing effects of stress in monkeys performing a spatial working memory task.

FIG. 2 demonstrates that a low dose of the selective α1A-AR antagonist, RS17053 (RS17), blocks the impairing effects of stress in monkeys performing a spatial working memory task, in accordance with some embodiments. As depicted in FIG. 2, the performance in the spatial working memory task of monkeys exposed to stress or vehicle and administered with a low dose of RS17053 (RS17) (0.01 mg/kg) or vehicle (VEH). In this experiment, rhesus monkeys exposed to a pharmacological agent that induces a stress response were impaired in performing a spatial working memory task that relies on the prefrontal cortex (VEH+STRESS). However, if the monkeys received pretreatment with the low dose of RS17053 prior to the stressor, their cognitive performance was protected from stress exposure, performing as well (or even better) than under control, nonstress conditions. Results represent mean+SEM percent correct on the spatial working memory task. **=significantly different from vehicle control (“VEH+VEH”); §=significant difference from VEH+STRESS; VEH=vehicle. In certain embodiments, a low dose of the selective alpha1-AR antagonist RS17053 blocks the impairing effects of stress in monkeys performing a spatial working memory task.

FIG. 3 depicts the performance in the spatial working memory task of monkeys exposed to stress or vehicle and administered with 0.1 mg/kg silodosin (SIL) or vehicle (VEH). Reversal of stress-induced cognitive deficits with the selective alpha1A-AR antagonist, silodosin. Stress is known to impair the higher cognitive functions that rely on the prefrontal cortex in humans, monkeys and rodents. In this experiment, rhesus monkeys exposed to a pharmacological agent that induces a stress response were impaired in performing a spatial working memory task that relies on the prefrontal cortex (VEH+STRESS). However, if the monkeys received pretreatment with silodosin prior to the stressor, their cognitive performance was protected from stress exposure, performing as well as under control, nonstress conditions. Results represent mean+SEM percent correct on the spatial working memory task. VEH=vehicle, SIL=silodosin. In certain embodiments, the selective alpha1-AR antagonist silodosin blocks the impairing effects of stress in monkeys performing a spatial working memory task.

FIG. 4 depicts the performance in the spatial working memory task of monkeys exposed to stress or vehicle and administered with 0.1 mg/kg tamulosin (TAM) or vehicle (VEH). Reversal of stress-induced cognitive deficits with the selective alpha1A-AR antagonist, tamulosin. Stress is known to impair the higher cognitive functions that rely on the prefrontal cortex in humans, monkeys and rodents. In this experiment, rhesus monkeys exposed to a pharmacological agent that induces a stress response were impaired in performing a spatial working memory task that relies on the prefrontal cortex (VEH+STRESS). However, if the monkeys received pretreatment with tamulosin prior to the stressor, their cognitive performance was protected from stress exposure, performing as well as under control, nonstress conditions. VEH=vehicle, TAM=tamulosin. In certain embodiments, the selective alpha1-AR antagonist tamulosin blocks the impairing effects of stress in monkeys performing a spatial working memory task.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

In human, as well as some other mammals, three subtypes of alpha-1-adrenoceptor exist: the α1A-adrenergic receptor subtype (α1A-AR), the α1B-adrenergic receptor subtype (α1B-AR), and the α1D-adrenergic receptor subtype (α1D-AR). In certain non-limiting embodiments, there has been no research on the role or importance of the individual subtypes of alpha-1-adrenoceptor in mediating the stress response.

Without wishing to be bound by theory, the various α1-adrenergic receptor subtypes have distinct effects on the stress response. In certain embodiments, the compounds contemplated in the invention are selective antagonists of the α1A-adrenergic receptor subtype(s) that play a role in stress disorders. In certain embodiments, the compounds contemplated in the invention are weaker antagonists (or have negligible antagonistic activity) against one or more other α1-adrenergic receptor subtypes that do not play a role in stress disorders. In certain embodiments, the compounds contemplated in the invention are weaker antagonists (or have negligible antagonistic activity) against one or more other α1-adrenergic receptor subtypes that are associated with undesirable side effects.

Without wishing to be bound by theory, the undesirable results of prazosin and doxazosin in treating stress-related disorders are, at least partly, due to the fact that these compounds lack specificity for the α1A-adrenergic receptor subtype.

As previously reported (Kenny et al., Br J Pharmacol. 1996 Jun.; 118(4):871-8), the binding affinities of prazosin for cloned human α1A, α1B and α1D adrenergic receptors are, as measured according to the inhibition constant (pKi), 9.7±0.20, 9.6±0.14, and 9.5±0.10, respectively. The pKi values of doxazosin for cloned human α1A, α1B and α1D adrenergic receptors are 8.5±0.20, 9.0±0.20, and 8.4±0.12, respectively. Therefore, prazosin blocks all alpha-1-adrenoceptor subtypes equally strong, and doxazosin has a preference for the α1B subtype, rather than the α1A subtype.

As demonstrated herein, antagonists specific for the α1A-adrenergic receptor subtype can effectively treat stress-related disorders, causing fewer side effects than non-specific α1-adrenergic receptor subtype antagonists.

Antagonists specific for α1A-adrenergic receptor subtype are known in the art. Alpha-1A-adrenoceptor antagonists were approved by the U.S. Food and Drug Administration (FDA) as early as 1997 to treat benign prostatic hyperplasia (BPH). However, because that there has been no understanding in the art that alpha-1-adrenoceptor subtypes may have differential or even opposing effects on prefrontal function (and that selective blockade of the alpha-1A-adrenoceptor is thus be more effective than nonselective alpha-1-adrenoceptor antagonists), the antagonists specific for the α1A-adrenergic receptor subtype has not been experimented on to treat stress-related disorders, such as stress-induced prefrontal cortical dysfunction.

Experiments on treating stress-induced prefrontal deficits with two exemplary α1A-adrenergic receptor antagonists, RS17053 and tamsulosin, were performed on rhesus monkeys. As previously reported (Kenny et al., Br J Pharmacol. 1996 Jun.;118(4):871-8), RS17053 has a strong preference for α1A-AR (pKi for cloned human α1A-AR=8.6±0.09) over α1B-AR (pKi for cloned human α1B-AR=7.3±0.09) and α1D-AR (pKi for cloned human α1D-AR=7.1±0.09), while tamsulosin has a preference for α1A-AR and α1D-AR over α1B-AR. As demonstrated herein, both compounds were found to be effective for treating disorders consequent of stress. Notably, as described herein, the α1A-AR specific RS17053 completely blocked the stress-induced prefrontal deficits in the rhesus monkeys. These results show that selective α1A-adrenergic receptor subtype antagonist can be used to treat stress-related disorders.

Accordingly, the instant specification is directed to method of treating, ameliorating, and/or preventing a stress-related disorder in a subject in need thereof. In certain embodiments, the method includes administering to the subject an effective amount of a α1A-adrenergic receptor subtype specific inhibitor.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of ±20% or ±10%, in certain embodiments ±5%, in certain embodiments ±1%, in certain embodiments ±0.1% from the specified value, as such variations are appropriate to perform the disclosed methods.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “patient,” “individual” or “subject” refers to a human or a non-human mammal. Non-human mammals include, for example, livestock and pets, such as ovine, bovine, porcine, canine, feline and murine mammals. In certain embodiments, the patient, individual or subject is human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt or solvate of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof. Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “treatment” or “treating” is defined as the application or administration of a therapeutic agent, i.e., a compound useful within the invention (alone or in combination with another pharmaceutical agent), to a patient, or application or administration of a therapeutic agent to an isolated tissue or cell line from a patient (e.g., for diagnosis or ex vivo applications), who has a disease or disorder, and/or a symptom of a disease or disorder, with the purpose to cure, heal, alleviate, relieve, alter, remedy, ameliorate, improve, or affect the disease or disorder, and/or the symptoms of the disease or disorder. Such treatments may be specifically tailored or modified, based on knowledge obtained from the field of pharmacogenomics.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Method of Treating, Ameliorating, and/or Preventing a Stress-Related Disorder

In some embodiments, the instant specification is directed to a method of treating, ameliorating, and/or preventing a stress-related disorder in a subject in need thereof. In some embodiments, the method includes administering to the subject a therapeutically effective amount of a selective α1-adrenergic receptor antagonist.

In some embodiments, the inhibition constant (pKi) of the selective α1-adrenergic receptor antagonist for α1A-adrenergic receptor subtype (α1A-AR) is larger than the pKi of the selective α1-adrenergic receptor antagonist for α1B-adrenergic receptor subtype (α1B-AR) and/or the pKi of the selective α1-adrenergic receptor antagonist for α1D-adrenergic receptor subtype (α1D-AR).

In some embodiments, the compound contemplated in the invention has an inhibition constant (pKi) for the α1A-adrenergic receptor subtype (α1A-AR) that is about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0 units or higher than for the α1B-adrenergic receptor subtype (α1B-AR).

In some embodiments, the compound contemplated in the invention has an inhibition constant (pKi) for the α1A-adrenergic receptor subtype (α1A-AR) that is about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0 units or higher than for the α1D-adrenergic receptor subtype (α1D-AR).

In some embodiments, the compound contemplated in the invention has an inhibition constant (pKi) for the α1A-adrenergic receptor subtype (α1A-AR) about 7.0 units or higher, such as about 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9 or 11.0 units or higher.

In some embodiments, the compound contemplated in the invention is selective toward α1A-adrenergic receptor subtype (α1A-AR) over the α1B-adrenergic receptor subtype (α1B-AR) and/or the α1A-adrenergic receptor subtype (α1A-AR).

In some embodiments, the compound contemplated in the invention is about three times or more selective for the α1A-adrenergic receptor subtype (α1A-AR) than for the α1B-adrenergic receptor subtype (α1B-AR), such as about 5 times or more, 10 times or more, 20 times or more, 50 times or more, 75 times or more, 100 times or more, 200 times or more, 500 times or more, 1000 times or more selective for the α1A-adrenergic receptor subtype (α1A-AR) than for the α1B-adrenergic receptor subtype (α1B-AR).

In some embodiments, the compound contemplated in the invention is about three times or more selective for the α1A-adrenergic receptor subtype (α1A-AR) than for the α1D-adrenergic receptor subtype (α1D-AR), such as about 5 times or more, 10 times or more, 20 times or more, 50 times or more, 75 times or more, 100 times or more, 200 times or more, 500 times or more, 1000 times or more selective for the α1A-adrenergic receptor subtype (α1A-AR) than for the α1D-adrenergic receptor subtype (α1D-AR).

In some embodiments, the selective α1A-adrenergic receptor antagonist includes indoramin (also known as N-{1-[2-(1H-indol-3-yl)ethyl]piperidin-4-yl} benzamide).

In some embodiments, the selective α1A-adrenergic receptor antagonist includes Rec 15/2739 (also known as 1-(4-amino-6,7-dimethoxy-2-quinazolinyl)-4-[[2-methoxy-6-(1-methylethyl)phenoxy]acetyl]piperazine; pKi value of 9.0±0.07 for α1A-AR, pKi value of 7.5±0.06 for α1B-AR, pKi value of 8.6±0.07 for α1D-AR, Kenny et al., Br J Pharmacol. 1996 Jun.;118(4):871-8).

In some embodiments, the selective α1A-adrenergic receptor antagonist includes RS-17053 (also known as (N-[2-(2-cyclopropylmethoxyphenoxy)ethyl]-5-chloro-α,α-dimethyl-1H-indole-3-ethanamine); pKi value of 8.6±0.09 for α1A-AR, pKi value of 7.3±0.09 for α1B-AR, pKi value of 7.1±0.09 for α1D-AR, Kenny et al.).

In some embodiments, the selective α1A-adrenergic receptor antagonist includes RS 100329 hydrochloride (also known as 5-Methyl-3-[3-[3-[4-[2-(2,2,2,-trifluroethoxy)phenyl]-1-piperazinyl]propyl]-2,4-(1H,3H)-pyrimidinedione hydrochloride); pKi value of 9.6 for α1A-AR, with 126 and 50 fold selectivity over the alphaα1B-and alpha1D-AR subtypes, respectively. Williams et al. Br J Pharmacol. 1999 May;127(1):252-8).

In some embodiments, the selective α1A-adrenergic receptor antagonist includes silodosin (also known as 1-(3-hydroxypropyl)-5-[(2R)-({2-[2-[2-(2,2,2-trifluoroethoxy)phenoxy]ethyl}amino)propyl] indoline-7-carboxamide; pKi value of 10.4 for α1A-AR, pKi value of 8.19 for α1B-AR, pKi value of 8.66 for α1D-AR, CHMP ASSESSMENT REPORT FOR Silodyx, INN-silodosin-European Medicines Agency).

In some embodiments, the selective α1A-adrenergic receptor antagonist includes tamsulosin (also known as (5-(2-{[2-(2-Ethoxyphenoxy)ethyl]amino}propyl)-2-methoxybenzene-1-sulfonamide), such as (R)-tamsulosin or(S)-tamsulosin (pKi value of 8.4±0.06 for α1A-AR, pKi value of 7.0±0.08 for α1B-AR, pKi value of 8.1±0.04 for α1D-AR, Kenny et al.).

In some embodiments, the selective α1A-adrenergic receptor antagonist includes 5-methyl-urapidil (Gross et al., Eur J Pharmacol 1988; 151:333-5).

The compounds contemplated in the disclosure may possess one or more stereocenters, and each stereocenter may exist independently in either the (R) or(S) configuration. In certain embodiments, compounds described herein are present in optically active or racemic forms. The compounds described herein encompass racemic, optically active, regioisomeric and stereoisomeric forms, or combinations thereof that possess the therapeutically useful properties described herein. Preparation of optically active forms is achieved in any suitable manner, including, by way of non-limiting example, by resolution of the racemic form with recrystallization techniques, synthesis from optically active starting materials, chiral synthesis, or chromatographic separation using a chiral stationary phase. A compound illustrated herein by the racemic formula further represents either of the two enantiomers or any mixtures thereof, or in the case where two or more chiral centers are present, all diastereomers or any mixtures thereof.

In certain embodiments, the compounds contemplated in the disclosure exist as tautomers. All tautomers are included within the scope of the compounds recited herein.

Compounds described herein also include isotopically labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, substitution with heavier isotopes such as deuterium affords greater chemical stability. Isotopically labeled compounds are prepared by any suitable method or by processes using an appropriate isotopically labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

In all of the embodiments provided herein, examples of suitable optional substituents are not intended to limit the scope of the claimed disclosure. The compounds of the disclosure may contain any of the substituents, or combinations of substituents, provided herein.

In some embodiments, the invention contemplates the use of any compound described herein, or a salt, solvate, enantiomer, enantiomeric mixture, diastereoisomer, diastereoisomeric mixture, geometric isomer, tautomer, and/or isotopically labelled isomer thereof, and/or any mixture thereof.

In some embodiments, the stress-related disorder is a stress-induced prefrontal cortical dysfunction.

In some embodiments, the stress-related disorder comprises post-traumatic stress disorder (PTSD). In some embodiments, the stress-related disorder comprises an anxiety disorder. In some embodiments, the stress-related disorder comprises an agitation/disruptive/aggressive behavior, including disruptive behaviors associated with dementia.

In some embodiments, a dosage of the selective α1-adrenergic receptor antagonist compound administered to the subject is equal to or greater than about 0.001 mg/kg. In some embodiments, a dosage of the selective α1-adrenergic receptor antagonist compound administered to the subject is equal to or greater than about 0.002 mg/kg. In some embodiments, a dosage of the selective α1-adrenergic receptor antagonist compound administered to the subject is equal to or greater than about 0.003 mg/kg. In some embodiments, a dosage of the selective α1-adrenergic receptor antagonist compound administered to the subject is equal to or greater than about 0.004 mg/kg. In some embodiments, a dosage of the selective α1-adrenergic receptor antagonist compound administered to the subject is equal to or greater than about 0.005 mg/kg. In some embodiments, a dosage of the selective α1-adrenergic receptor antagonist compound administered to the subject is equal to or greater than about 0.006 mg/kg. In some embodiments, a dosage of the selective α1-adrenergic receptor antagonist compound administered to the subject is equal to or greater than about 0.007 mg/kg. In some embodiments, a dosage of the selective α1-adrenergic receptor antagonist compound administered to the subject is equal to or greater than about 0.008 mg/kg. In some embodiments, a dosage of the selective α1-adrenergic receptor antagonist compound administered to the subject is equal to or greater than about 0.009 mg/kg. In some embodiments, a dosage of the selective α1-adrenergic receptor antagonist compound administered to the subject is equal to or greater than about 0.01 mg/kg. In some embodiments, a dosage of the selective α1-adrenergic receptor antagonist compound administered to the subject is equal to or greater than about 0.02 mg/kg. In some embodiments, a dosage of the selective α1-adrenergic receptor antagonist compound administered to the subject is equal to or greater than about 0.03 mg/kg. In some embodiments, a dosage of the selective α1-adrenergic receptor antagonist compound administered to the subject is equal to or greater than about 0.04 mg/kg. In some embodiments, a dosage of the selective α1-adrenergic receptor antagonist compound administered to the subject is equal to or greater than about 0.05 mg/kg. In some embodiments, a dosage of the selective α1-adrenergic receptor antagonist compound administered to the subject is equal to or greater than about 0.06 mg/kg. In some embodiments, a dosage of the selective α1-adrenergic receptor antagonist compound administered to the subject is equal to or greater than about 0.07 mg/kg. In some embodiments, a dosage of the selective α1-adrenergic receptor antagonist compound administered to the subject is equal to or greater than about 0.08 mg/kg. In some embodiments, a dosage of the selective α1-adrenergic receptor antagonist compound administered to the subject is equal to or greater than about 0.09 mg/kg. In some embodiments, a dosage of the selective α1-adrenergic receptor antagonist compound administered to the subject is equal to or greater than about 0.1 mg/kg. In some embodiments, a dosage of the selective α1-adrenergic receptor antagonist compound administered to the subject is equal to or greater than about 0.2 mg/kg. In some embodiments, a dosage of the selective α1-adrenergic receptor antagonist compound administered to the subject is equal to or greater than about 0.3 mg/kg. In some embodiments, a dosage of the selective α1-adrenergic receptor antagonist compound administered to the subject is equal to or greater than about 0.4 mg/kg. In some embodiments, a dosage of the selective α1-adrenergic receptor antagonist compound administered to the subject is equal to or greater than about 0.5 mg/kg. In some embodiments, a dosage of the selective α1-adrenergic receptor antagonist compound administered to the subject is equal to or greater than about 0.6 mg/kg. In some embodiments, a dosage of the selective α1-adrenergic receptor antagonist compound administered to the subject is equal to or greater than about 0.7 mg/kg. In some embodiments, a dosage of the selective α1-adrenergic receptor antagonist compound administered to the subject is equal to or greater than about 0.8 mg/kg. In some embodiments, a dosage of the selective α1-adrenergic receptor antagonist compound administered to the subject is equal to or greater than about 0.9 mg/kg. In some embodiments, a dosage of the selective α1-adrenergic receptor antagonist compound administered to the subject is equal to or greater than about 1 mg/kg.

In some embodiments, a dosage of the selective α1-adrenergic receptor antagonist compound administered to the subject is equal to or less than about 0.001 mg/kg. In some embodiments, a dosage of the selective α1-adrenergic receptor antagonist compound administered to the subject is equal to or less than about 0.002 mg/kg. In some embodiments, a dosage of the selective α1-adrenergic receptor antagonist compound administered to the subject is equal to or less than about 0.003 mg/kg. In some embodiments, a dosage of the selective α1-adrenergic receptor antagonist compound administered to the subject is equal to or less than about 0.004 mg/kg. In some embodiments, a dosage of the selective α1-adrenergic receptor antagonist compound administered to the subject is equal to or less than about 0.005 mg/kg. In some embodiments, a dosage of the selective α1-adrenergic receptor antagonist compound administered to the subject is equal to or less than about 0.006 mg/kg. In some embodiments, a dosage of the selective α1-adrenergic receptor antagonist compound administered to the subject is equal to or less than about 0.007 mg/kg. In some embodiments, a dosage of the selective α1-adrenergic receptor antagonist compound administered to the subject is equal to or less than about 0.008 mg/kg. In some embodiments, a dosage of the selective α1-adrenergic receptor antagonist compound administered to the subject is equal to or less than about 0.009 mg/kg. In some embodiments, a dosage of the selective α1-adrenergic receptor antagonist compound administered to the subject is equal to or less than about 0.01 mg/kg. In some embodiments, a dosage of the selective α1-adrenergic receptor antagonist compound administered to the subject is equal to or less than about 0.02 mg/kg. In some embodiments, a dosage of the selective α1-adrenergic receptor antagonist compound administered to the subject is equal to or less than about 0.03 mg/kg. In some embodiments, a dosage of the selective α1-adrenergic receptor antagonist compound administered to the subject is equal to or less than about 0.04 mg/kg. In some embodiments, a dosage of the selective α1-adrenergic receptor antagonist compound administered to the subject is equal to or less than about 0.05 mg/kg. In some embodiments, a dosage of the selective α1-adrenergic receptor antagonist compound administered to the subject is equal to or less than about 0.06 mg/kg. In some embodiments, a dosage of the selective α1-adrenergic receptor antagonist compound administered to the subject is equal to or less than about 0.07 mg/kg. In some embodiments, a dosage of the selective α1-adrenergic receptor antagonist compound administered to the subject is equal to or less than about 0.08 mg/kg. In some embodiments, a dosage of the selective α1-adrenergic receptor antagonist compound administered to the subject is equal to or less than about 0.09 mg/kg. In some embodiments, a dosage of the selective α1-adrenergic receptor antagonist compound administered to the subject is equal to or less than about 0.1 mg/kg. In some embodiments, a dosage of the selective α1-adrenergic receptor antagonist compound administered to the subject is equal to or less than about 0.2 mg/kg. In some embodiments, a dosage of the selective α1-adrenergic receptor antagonist compound administered to the subject is equal to or less than about 0.3 mg/kg. In some embodiments, a dosage of the selective α1-adrenergic receptor antagonist compound administered to the subject is equal to or less than about 0.4 mg/kg. In some embodiments, a dosage of the selective α1-adrenergic receptor antagonist compound administered to the subject is equal to or less than about 0.5 mg/kg. In some embodiments, a dosage of the selective α1-adrenergic receptor antagonist compound administered to the subject is equal to or less than about 0.6 mg/kg. In some embodiments, a dosage of the selective α1-adrenergic receptor antagonist compound administered to the subject is equal to or less than about 0.7 mg/kg. In some embodiments, a dosage of the selective α1-adrenergic receptor antagonist compound administered to the subject is equal to or less than about 0.8 mg/kg. In some embodiments, a dosage of the selective α1-adrenergic receptor antagonist compound administered to the subject is equal to or less than about 0.9 mg/kg. In some embodiments, a dosage of the selective α1-adrenergic receptor antagonist compound administered to the subject is equal to or less than about 1 mg/kg.

In some embodiments, the dosage of the selective α1-adrenergic receptor antagonist compound administered to the subject is equal to or less than about 1 mg/kg. In some embodiments, the dosage of the selective α1-adrenergic receptor antagonist compound administered to the subject is equal to or less than about 0.9 mg/kg. In some embodiments, the dosage of the selective α1-adrenergic receptor antagonist compound administered to the subject is equal to or less than about 0.8 mg/kg. In some embodiments, the dosage of the selective α1-adrenergic receptor antagonist compound administered to the subject is equal to or less than about 0.7 mg/kg. In some embodiments, the dosage of the selective α1-adrenergic receptor antagonist compound administered to the subject is equal to or less than about 0.6 mg/kg. In some embodiments, the dosage of the selective α1-adrenergic receptor antagonist compound administered to the subject is equal to or less than about 0.5 mg/kg. In some embodiments, the dosage of the selective α1-adrenergic receptor antagonist compound administered to the subject is equal to or less than about 0.4 mg/kg. In some embodiments, the dosage of the selective α1-adrenergic receptor antagonist compound administered to the subject is equal to or less than about 0.3 mg/kg. In some embodiments, the dosage of the selective α1-adrenergic receptor antagonist compound administered to the subject is equal to or less than about 0.2 mg/kg. In some embodiments, the dosage of the selective α1-adrenergic receptor antagonist compound administered to the subject is equal to or less than about 0.1 mg/kg. In some embodiments, the dosage of the selective α1-adrenergic receptor antagonist compound administered to the subject is equal to or less than about 0.09 mg/kg. In some embodiments, the dosage of the selective α1-adrenergic receptor antagonist compound administered to the subject is equal to or less than about 0.08 mg/kg. In some embodiments, the dosage of the selective α1-adrenergic receptor antagonist compound administered to the subject is equal to or less than about 0.07 mg/kg. In some embodiments, the dosage of the selective α1-adrenergic receptor antagonist compound administered to the subject is equal to or less than about 0.06 mg/kg. In some embodiments, the dosage of the selective α1-adrenergic receptor antagonist compound administered to the subject is equal to or less than about 0.05 mg/kg. In some embodiments, the dosage of the selective α1-adrenergic receptor antagonist compound administered to the subject is equal to or less than about 0.04 mg/kg. In some embodiments, the dosage of the selective α1-adrenergic receptor antagonist compound administered to the subject is equal to or less than about 0.03 mg/kg. In some embodiments, the dosage of the selective α1-adrenergic receptor antagonist compound administered to the subject is equal to or less than about 0.02 mg/kg. In some embodiments, the dosage of the selective α1-adrenergic receptor antagonist compound administered to the subject is equal to or less than about 0.01 mg/kg. In some embodiments, the dosage of the selective α1-adrenergic receptor antagonist compound administered to the subject is equal to or less than about 0.009 mg/kg. In some embodiments, the dosage of the selective α1-adrenergic receptor antagonist compound administered to the subject is equal to or less than about 0.008 mg/kg. In some embodiments, the dosage of the selective α1-adrenergic receptor antagonist compound administered to the subject is equal to or less than about 0.007 mg/kg. In some embodiments, the dosage of the selective α1-adrenergic receptor antagonist compound administered to the subject is equal to or less than about 0.006 mg/kg. In some embodiments, the dosage of the selective α1-adrenergic receptor antagonist compound administered to the subject is equal to or less than about 0.005 mg/kg. In some embodiments, the dosage of the selective α1-adrenergic receptor antagonist compound administered to the subject is equal to or less than about 0.004 mg/kg. In some embodiments, the dosage of the selective α1-adrenergic receptor antagonist compound administered to the subject is equal to or less than about 0.003 mg/kg. In some embodiments, the dosage of the selective α1-adrenergic receptor antagonist compound administered to the subject is equal to or less than about 0.002 mg/kg. In some embodiments, the dosage of the selective α1-adrenergic receptor antagonist compound administered to the subject is equal to or less than about 0.001 mg/kg.

In some embodiments, the dosage of the selective α1-adrenergic receptor antagonist compound administered to the subject is equal to or greater than about 1 mg/kg. In some embodiments, the dosage of the selective α1-adrenergic receptor antagonist compound administered to the subject is equal to or greater than about 0.9 mg/kg. In some embodiments, the dosage of the selective α1-adrenergic receptor antagonist compound administered to the subject is equal to or greater than about 0.8 mg/kg. In some embodiments, the dosage of the selective α1-adrenergic receptor antagonist compound administered to the subject is equal to or greater than about 0.7 mg/kg. In some embodiments, the dosage of the selective α1-adrenergic receptor antagonist compound administered to the subject is equal to or greater than about 0.6 mg/kg. In some embodiments, the dosage of the selective α1-adrenergic receptor antagonist compound administered to the subject is equal to or greater than about 0.5 mg/kg. In some embodiments, the dosage of the selective α1-adrenergic receptor antagonist compound administered to the subject is equal to or greater than about 0.4 mg/kg. In some embodiments, the dosage of the selective α1-adrenergic receptor antagonist compound administered to the subject is equal to or greater than about 0.3 mg/kg. In some embodiments, the dosage of the selective α1-adrenergic receptor antagonist compound administered to the subject is equal to or greater than about 0.2 mg/kg. In some embodiments, the dosage of the selective α1-adrenergic receptor antagonist compound administered to the subject is equal to or greater than about 0.1 mg/kg. In some embodiments, the dosage of the selective α1-adrenergic receptor antagonist compound administered to the subject is equal to or greater than about 0.09 mg/kg. In some embodiments, the dosage of the selective α1-adrenergic receptor antagonist compound administered to the subject is equal to or greater than about 0.08 mg/kg. In some embodiments, the dosage of the selective α1-adrenergic receptor antagonist compound administered to the subject is equal to or greater than about 0.07 mg/kg. In some embodiments, the dosage of the selective α1-adrenergic receptor antagonist compound administered to the subject is equal to or greater than about 0.06 mg/kg. In some embodiments, the dosage of the selective α1-adrenergic receptor antagonist compound administered to the subject is equal to or greater than about 0.05 mg/kg. In some embodiments, the dosage of the selective α1-adrenergic receptor antagonist compound administered to the subject is equal to or greater than about 0.04 mg/kg. In some embodiments, the dosage of the selective α1-adrenergic receptor antagonist compound administered to the subject is equal to or greater than about 0.03 mg/kg. In some embodiments, the dosage of the selective α1-adrenergic receptor antagonist compound administered to the subject is equal to or greater than about 0.02 mg/kg. In some embodiments, the dosage of the selective α1-adrenergic receptor antagonist compound administered to the subject is equal to or greater than about 0.01 mg/kg. In some embodiments, the dosage of the selective α1-adrenergic receptor antagonist compound administered to the subject is equal to or greater than about 0.009 mg/kg. In some embodiments, the dosage of the selective α1-adrenergic receptor antagonist compound administered to the subject is equal to or greater than about 0.008 mg/kg. In some embodiments, the dosage of the selective α1-adrenergic receptor antagonist compound administered to the subject is equal to or greater than about 0.007 mg/kg. In some embodiments, the dosage of the selective α1-adrenergic receptor antagonist compound administered to the subject is equal to or greater than about 0.006 mg/kg. In some embodiments, the dosage of the selective α1-adrenergic receptor antagonist compound administered to the subject is equal to or greater than about 0.005 mg/kg. In some embodiments, the dosage of the selective α1-adrenergic receptor antagonist compound administered to the subject is equal to or greater than about 0.004 mg/kg. In some embodiments, the dosage of the selective α1-adrenergic receptor antagonist compound administered to the subject is equal to or greater than about 0.003 mg/kg. In some embodiments, the dosage of the selective α1-adrenergic receptor antagonist compound administered to the subject is equal to or greater than about 0.002 mg/kg. In some embodiments, the dosage of the selective α1-adrenergic receptor antagonist compound administered to the subject is equal to or greater than about 0.001 mg/kg.

### Combination Therapies

The compounds of the present invention are intended to be useful in the methods of present invention in combination with one or more additional compounds useful for treating, ameliorating, and/or preventing any of the diseases or disorders contemplated within the invention. These additional compounds may comprise compounds of the present invention or compounds, e.g., commercially available compounds, known to treat, prevent, or reduce the symptoms of the diseases or disorders contemplated within the invention.

A synergistic effect may be calculated, for example, using suitable methods such as, for example, the Sigmoid-Eₘₐₓ equation (Holford & Scheiner, 19981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the patient either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions useful within the present invention to a patient, preferably a mammal, more preferably a human, may be carried out using known procedures, at dosages and for periods of time effective to treat a disease or disorder in the patient. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder in a patient.

In certain embodiments, the compositions useful within the invention are formulated using one or more pharmaceutically acceptable excipients or carriers. In certain embodiments, the pharmaceutical compositions of the invention comprise a therapeutically effective amount of a compound useful within the invention and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions useful within the invention are administered to the patient in dosages that range from one to five times per day or more. In other embodiments, the compositions useful within the invention are administered to the patient in range of dosages that include, but are not limited to, once every day, every two, days, every three days to once a week, and once every two weeks. It will be readily apparent to one skilled in the art that the frequency of administration of the various combination compositions useful within the invention will vary from individual to individual depending on many factors including, but not limited to, age, disease or disorder to be treated, gender, overall health, and other factors. Thus, the invention should not be construed to be limited to any particular dosage regime and the precise dosage and composition to be administered to any patient will be determined by the attending physical taking all other factors about the patient into account.

Compounds for administration may be in the range of from about 1 mg to about 10,000 mg, about 20 mg to about 9,500 mg, about 40 mg to about 9,000 mg, about 75 mg to about 8,500 mg, about 150 mg to about 7,500 mg, about 200 mg to about 7,000 mg, about 3050 mg to about 6,000 mg, about 500 mg to about 5,000 mg, about 750 mg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments therebetween.

In certain embodiments, the dose of a compound is from about 1 mg and about 2,500 mg. In other embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in certain embodiments, a dose of a second compound (i.e., a drug used for treating a disease or disorder) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other cognition improving agents.

The term “container” includes any receptacle for holding the pharmaceutical composition. For example, in certain embodiments, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a disease or disorder in a patient.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

Non-limiting examples of formulations useful within the invention, including formulations of prodrugs of compounds useful within the invention, are recited in the following patent application publications, each of which is incorporated by reference in its entirety herein: US 2012/0065152; WO 2011/033296; US 2011/0065796; and WO 2007/016284.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e. having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e. drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present invention also includes a multi-layer tablet comprising a layer providing for the delayed release of one or more compounds of the invention, and a further layer providing for the immediate release of a medication for treatment of a disease or disorder. Using a wax/pH-sensitive polymer mix, a gastric insoluble composition may be obtained in which the active ingredient is entrapped, ensuring its delayed release.

### Parenteral Administration

For parenteral administration, the compounds may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this invention include dosage forms as described in U.S. Pat. Nos. 6,340,475, 6,488,962, 6,451,808, 5,972,389, 5,582,837, and 5,007,790. Additional dosage forms of this invention also include dosage forms as described in U.S. Patent Applications Nos. 20030147952, 20030104062, 20030104053, 20030044466, 20030039688, and 20020051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In a preferred embodiment of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound will depend on the age, sex and weight of the patient, the current medical condition of the patient and the progression of cognitive changes in the patient being treated. The skilled artisan will be able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### Examples

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

Example 1: Selective alpha1A-AR antagonists block the impairing effects of stress in monkeys performing a spatial working memory task

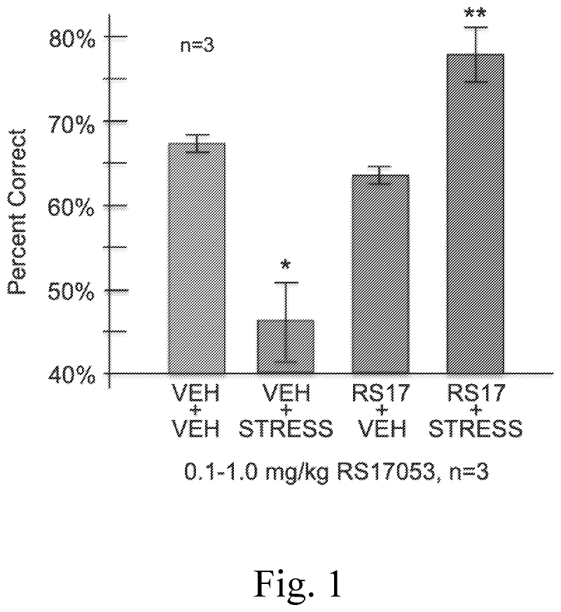

Rhesus monkeys (Macaca mulatta) exposed to stress or vehicle and administered with RS17053 (“RS17”) or vehicle (“VEH”) (FIG. 1) or administered with silodosin (“SIL”) or vehicle (“VEH”) were subjected to the oculomotor delayed response (ODR) tasks similar to those as described previously (Datta et al., J Neurosci. 2019 Apr. 3;39(14):2722-2734, the entirety of the journal article is hereby incorporated herein by reference). Specifically, the experiments were performed as the follows:

The monkeys were seated in primate chairs with their heads fixed and faced a 27-inch computer monitor 30 inches in front of them. The monkeys' eye positions were monitored with the ISCAN Eye Movement Monitoring System. The monkeys were pretrained on the visuospatial ODRtask, which required the subject to make a memory-guided saccade to a remembered visuospatial target. The ODR task was generated by the PICTO system (custom-designed, Windows-based data acquisition software). A central small white circle was illuminated on the computer monitor, which served as the fixation target. To initiate a trial, the animal fixated this central target and maintained fixation for 0.5 s (fixation period), whereupon a cue (the same sized white circle) was illuminated for a period of 0.5 s (cue period) at 1 of 8 peripheral targets located at an eccentricity of 13° with respect to the fixation spot. After the cue was extinguished, a 2.5 s delay period followed. The subject was required to maintain central fixation throughout both the cue presentation and the delay period. At the end of the delay, the fixation spot was extinguished, which instructed the monkey to make a memory-guided saccade to the location where the cue had been shown before the delay period. A trial was considered successful if the subject's response was completed within 0.5 s of the offset of the fixation spot and was within 2° around the correct cue location. The subject was rewarded with fruit juice immediately after every successful response. The position of the stimulus was randomized over trials such that it had to be remembered on a triα1-by-trial basis. The intertrial intervals (ITIs) were at least 3 s. The subject performed 1000-1500 trials per session.

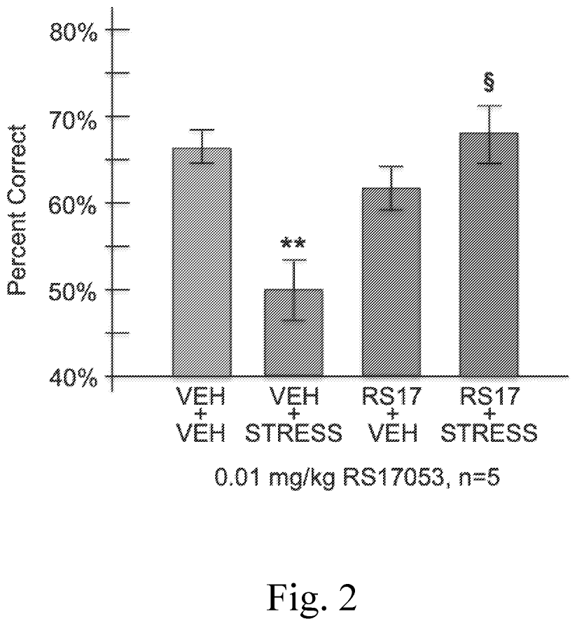

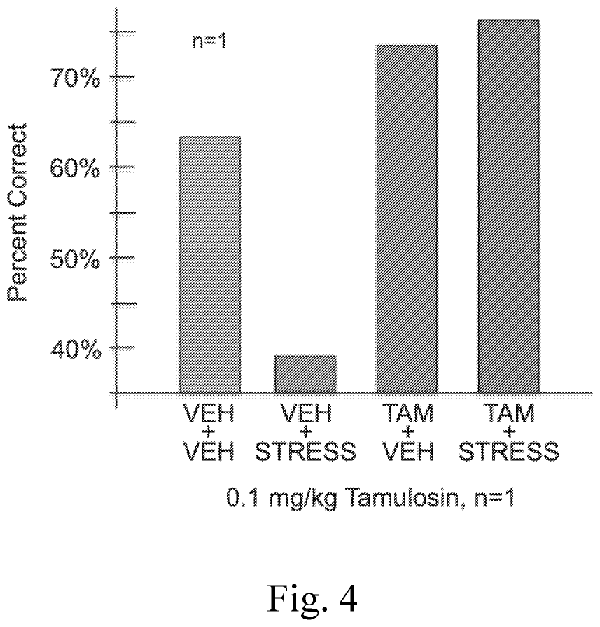

Referring to FIGS. 1-4, as expected, stress significantly decreases the success rate of performing the spatial working memory task in monkeys that are not treated with RS17053 silodosin, or tamulosin (“VEH+VEH” vs. “VEH+STRESS”). While RS17053 has no significant effect in monkeys that are not subjected to stress (“VEH+VEH” vs. “RS17+VEH” or “SIL+VEH”), all three α1A-adrenergic receptor specific antagonists completely blocked the stress-induced prefrontal deficits in the rhesus monkeys (“VEH+VEH” vs. “VEH+STRESS” vs. “RS17+STRESS,” “SIL+STRESS” or “TAM+STRESS”).

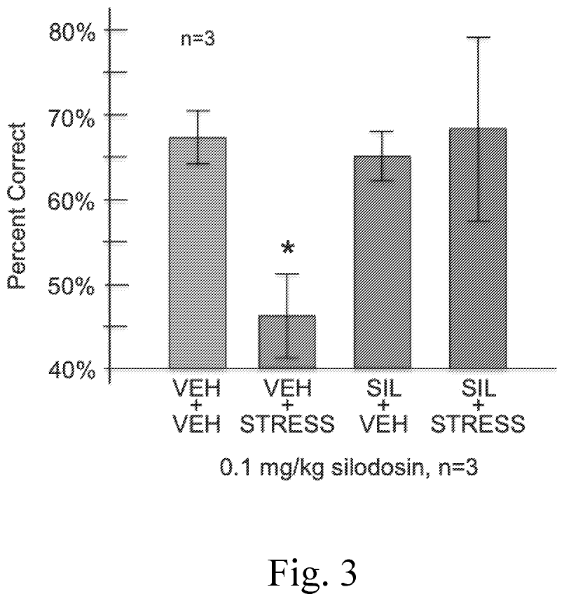

Referring to FIG. 3, it is worth noting that RS17053 was able to blocked the stress-induced prefrontal deficits in the rhesus monkeys stress at a dosage as low as 0.01 mg/kg.

### ENUMERATED EMBODIMENTS

In some aspects, the present invention is directed to the following non-limiting embodiment:
Embodiment 1: A method of treating, ameliorating, and/or preventing a stress-related disorder in a subject in need thereof, the method comprising administering to the subject a therapeutically effective amount of a selective α1-adrenergic receptor antagonist compound, wherein the compound has a larger inhibition constant (pKi) for the α1A-adrenergic receptor subtype (α1A-AR) than for the α1B-adrenergic receptor subtype (α1B-AR) and/or α1D-adrenergic receptor subtype (α1D-AR).
Embodiment 2: The method of Embodiment 1, wherein the compound has an inhibition constant (pKi) for the α1A-adrenergic receptor subtype (α1A-AR) that is 0.3 units or higher than for the α1B-adrenergic receptor subtype (α1B-AR) and/or α1D-adrenergic receptor subtype (α1D-AR).
Embodiment 3: The method of any one of Embodiments 1-2, wherein the selective α1A-adrenergic receptor antagonist compound comprises at least one selected from the group consisting of RS-17053, silodosin, 5-methyl-urapidil and tamsulosin.
Embodiment 4: The method of any one of Embodiments 1-3, wherein the stress-related disorder comprises a stress-induced prefrontal cortical dysfunction.
Embodiment 5: The method of any one of Embodiments 1-4, wherein the stress-related disorder is at least one selected form the group consisting of post-traumatic stress disorder (PTSD), an anxiety disorder, and an agitation/disruptive/aggressive behavior associated with dementia.
Embodiment 6: The method of any one of Embodiments 1-5, wherein the compound is administered by at least one route selected from the group consisting of oral, nasal, rectal, intravaginal, parenteral, buccal, sublingual, and topical.
Embodiment 7: The method of any one of Embodiments 1-6, wherein a dosage of the selective α1-adrenergic receptor antagonist administered to the subject is about 0.001 mg/kg or higher.
Embodiment 8: The method of any one of Embodiments 1-7, wherein a dosage of the selective α1-adrenergic receptor antagonist administered to the subject is about 1 mg/kg or lower.
Embodiment 9: The method of any one of Embodiments 1-8, wherein the subject is a mammal.
Embodiment 10: The method of any one of Embodiments 1-9, wherein the subject is a human.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

1. A method of treating, ameliorating, or preventing a stress-related disorder in a subject in need thereof, the method comprising administering to the subject a therapeutically effective amount of a selective α1A-adrenergic receptor antagonist compound, wherein the compound has a larger inhibition constant (pKi) for the α1A-adrenergic receptor subtype (α1A-AR) than for the α1B-adrenergic receptor subtype (α1B-AR) or α1D-adrenergic receptor subtype (α1D-AR).

2. The method of claim 1, wherein the compound has an inhibition constant (pKi) for the α1A-adrenergic receptor subtype (α1A-AR) that is 0.3 units or higher than for the α1B-adrenergic receptor subtype (α1B-AR) or α1D-adrenergic receptor subtype (α1D-AR).

3. The method of claim 1, wherein the selective α1A-adrenergic receptor antagonist compound comprises at least one selected from the group consisting of RS-17053, silodosin, 5-methyl-urapidil and tamsulosin.

4. The method of claim 1, wherein the stress-related disorder comprises a stress-induced prefrontal cortical dysfunction.

5. The method of claim 1, wherein the stress-related disorder is at least one selected form the group consisting of post-traumatic stress disorder (PTSD), an anxiety disorder, and an agitation/disruptive/aggressive behavior associated with dementia.

6. The method of claim 1, wherein the compound is administered by at least one route selected from the group consisting of oral, nasal, rectal, intravaginal, parenteral, buccal, sublingual, and topical.

7. The method of claim 1, wherein a dosage of the selective α1-adrenergic receptor antagonist administered to the subject is about 0.001 mg/kg or higher.

8. The method of claim 1, wherein a dosage of the selective α1-adrenergic receptor antagonist administered to the subject is about 1 mg/kg or lower.

9. The method of claim 1, wherein the subject is a mammal.

10. The method of claim 1, wherein the subject is a human.
